(19) United States
(12) Patent Application Publication
(10) Pub. No.: US 2005/0107755 A1
(43) Pub. Date: May 19, 2005

(54) Hand-held device enabling accurate dispensing of a drop of a liquid into the eye of a subject

(76) Inventors: Steven T. Berger, Longmeadow, MA (US); Richard P. Gibralter, Scarsdale, NY (US)

Correspondence Address: ALIX YALE & RISTAS LLP, 750 MAIN STREET, SUITE 1400, HARTFORD, CT, 06103, US

(21) Appl. No.: 10/713,890

(22) Filed: Nov. 14, 2003

(51) Int. Cl. A61M 35/00

(52) U.S. Cl. 604/295

(57) ABSTRACT

A system for delivering a drop of liquid to an eye of a subject, wherein the instrument has a receiver to receive a liquid to be delivered to the eye of the subject, a transfer portion to receive the liquid from the receiver to advance a drop of the liquid by gravity and capillary action through a capillary tube to a lower discharge outlet whereat the drop of liquid breaks away and drops from the capillary tube, and a lid retractor supported adjacent to the discharge outlet of the capillary tube, for being pressed against the lower lid of the subject to form a cul-de-sac at the lower lid. The lower discharge outlet of the capillary tube is positioned adjacent to and above the lid retractor in a position to deposit the drop of liquid into the cul-de-sac.

### FIELD OF THE INVENTION

The invention relates to an instrument and a method by which a drop of a liquid can be accurately administered by a person into his or her eye.

The invention particularly relates to such an instrument which can be operated with one hand of the person.

### BACKGROUND OF THE INVENTION

Many people throughout the world are required to administer liquid drops into their eyes for a variety of purposes, such as, for administering medication to control or prevent disease, to reduce inflammation, to reduce intraocular pressure, to supply liquid tears, etc.

Current practice involves dispensing the liquid from a container dropwise into the eye of the patient by squeezing the container.

However, particularly in the case of older and disabled people, accurate dispensing of the drops into the eye of the patient is unreliable. Often such patients are unsteady in holding the eye drop dispenser, have difficulty in tilting their head back or must lie in a supine position to allow the placement of the eye drop into the patient's eye. Frequently, more than one drop is dispensed and commonly the drops are not dispensed into the eye but land on the patient's cheek resulting in widespread wastage. In the case of very expensive medications, this is costly. In brief, standard squeezable eye drop dispensers for medications or liquid tears typically dispense more than one drop when the container is squeezed and often miss their target.

### SUMMARY OF THE INVENTION

An object of the present invention is to provide a device by which a drop of liquid will be dispensed accurately into the eye of the patient.

A further object of the invention is to provide such a device.which can be easily handled with one hand of the patient.

Another object of the invention is to provide such a device which enables the patient to apply traction to the lower lid of the eye to expose the inferior cul-de-sac of the lower lid into which the drop of liquid is accurately dispensed.

Yet another object of the invention is to provide such a device which is inexpensive and may be disposed of after a single or several uses.

Still another object of the invention is to provide such a device which is compact and is capable of being packaged so that a number of devices can be incorporated into a package and used one by one.

The above and further objects of the invention are achieved by a device which comprises a manually held instrument having a lid retractor and a liquid conveyor integrated and arranged so that liquid introduced into the liquid conveyor is advanced as a liquid drop and deposited into the eye of the subject whose lower lid is retracted by the lid retractor.

It is a feature of the invention that the instrument is constructed so that it can be held in one hand of the user to achieve the retraction of the lower lid and the dispensing of the liquid drop into the eye of the subject.

In further accordance with the invention, the instrument has a liquid conveyor which includes a receiver for the liquid to be delivered to the instrument and a transfer portion connected to the receiver to transfer the liquid to a capillary tube in which the liquid is advanced as a drop by gravity and capillary action to a lower discharge outlet of the capillary tube. Thereat, the drop of liquid breaks away and drops from the capillary tube into the inferior cul-de-sac of the lower lid of the eye which is exposed by a lid retractor supported adjacent to the discharge outlet of the capillary tube. The lid retractor retracts the lower lid of the eye when pressed against the lower lid to form the cul-de-sac of the lower lid.

The invention is characterized in that the lower discharge outlet of the capillary tube is positioned adjacent to and above the lid retractor so that when the liquid drop falls from the outlet of the capillary tube, it will fall directly and accurately into the cul-de-sac.

According to one embodiment of the invention (heretofore referred to as embodiment one), the receiver of the instrument is formed to engage and secure a conventional liquid dispenser therein so that the liquid can be introduced into the capillary tube by squeezing the liquid dispenser which remains attached to the instrument. The dispenser and the instrument can be removably stored in a sanitary case between individual dispensing of drops. The cover can be used for color coding or otherwise identifying the contents of the dispenser.

In accordance with another embodiment of the invention (heretofore referred to as embodiment two), it is contemplated that the instrument will be a single stand-alone article into which the eye liquid can be deposited from a conventional liquid dispenser which is then disconnected. The drop then flows to the discharge outlet whereat the lid retractor will have been pressed against the lower lid to retract the lid and expose the inferior cul-de-sac.

This embodiment two, will include a holder having finger-engaging means to facilitate the handling of the instrument and the dispensing of the liquid. In addition, it will incorporate a reservoir, which can receive a quantity of liquid such that when the attached flexible bulb is pressed or squeezed, a drop of liquid will be introduced into the capillary tube.

In another embodiment of the invention, the instrument can include a holder having finger-engaging means to facilitate the handling of the instrument and the dispensing of the liquid.

It is further contemplated according to the invention, to form the receiver as a loading chamber for the liquid to which is connected a flexible air-filled bulb such that when the bulb is pressed or squeezed a drop of liquid will be dispensed from the loading chamber into the capillary tube.

The invention also contemplates a method which utilizes the instrument for manual operation by one hand of the user such that when the instrument is pressed against the lower lid of the eye to expose the cul-de-sac of the lower lid, causing a drop of liquid to flow in the instrument and drop into the cul-de-sac.

It is a feature of the invention that in order to prevent reflux of the liquid, a one-way valve is provided.

It is a further feature of the invention that the lower ends of the capillary tube and the lid retractor are bent at an angle with respect to the longitudinal extent of the capillary tube in order to position the lid retractor to expose the cul-de-sac when pressed against the lower lid and to locate the discharge outlet of the bent end of the capillary tube to dispense the liquid drop into the cul-de-sac.

### BRIEF DESCRIPTION OF THE FIGURES OF THE DRAWING

FIG. 1 is a front view, partially in section, of a first embodiment of the device according to the invention in cooperation with a liquid dispenser;

FIG. 2 is a side view of the device and dispenser of FIG. 1;

FIG. 3 shows a part of the device of FIG. 1 in an operative position in which a drop of liquid is deposited into the eye of a subject;

FIG. 4a is a front view of the device of FIG. 1 shown in conjunction with an opened protective case;

FIG. 4b is a sectional view of the device and case in a closed position taken along the line A-A of FIG. 4a with an open case portion shown in phantom;

FIG. 5 is a front view of a modified device, embodiment two, shown in conjunction with the thumb and fingers of a user to illustrate the operation thereof; and

FIG. 6 is a perspective view of the modified device of FIG. 5.

### DETAILED DESCRIPTION

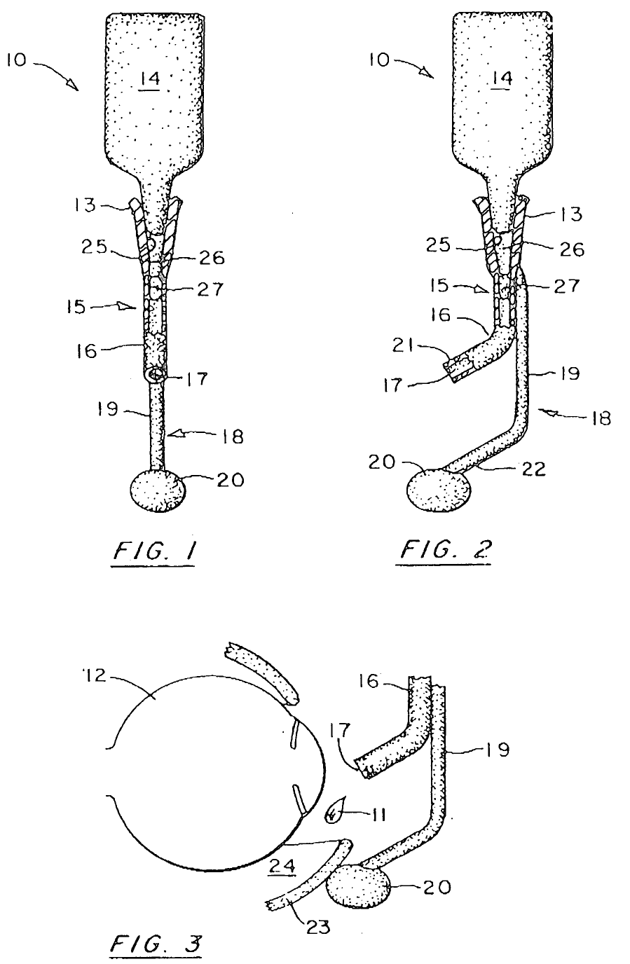

Referring to FIGS. 1-3, therein is shown a first embodiment (embodiment one) of an instrument 10 for delivering a drop of liquid 11 (FIG. 3) to an eye 12 of a user. The instrument 10 includes a receiver 13 of funnel shape adapted to receive the liquid from a dispenser 14. The dispenser 14 can be a conventional dispenser for an ophthalmic medication or for liquid tears, etc. By squeezing the dispenser 14, the drop of liquid is introduced into the receiver 13 of the instrument 10.

The receiver 13 is connected to a transfer portion 15 so that liquid dispensed into the receiver 13 will flow into the transfer portion 15. The transfer portion 15 includes a capillary tube 16 through which the liquid passes by capillary action and gravity to a slightly conical end with a lower discharge outlet 17. At the discharge outlet 17, the drop of liquid 11 breaks away and drops from the capillary tube as shown in FIG. 3.

Secured to the capillary tube is a lid retractor 18 which comprises a rod 19 secured to the capillary tube 16 and at the end of the rod is fixed a pressing member 20 made of a soft material which is adapted to contact and press against the lower lid of the eye of the user. An end portion 21 of the capillary tube and an end portion 22 of the lid retractor are bent at an angle to ensure accurate dispensing of the drop of liquid into the eye of the user.

In this respect, when the pressing member 20 is pressed against the lower lid 23 of the eye of the user, the lower lid is retracted to form a cul-de-sac 24 of the lower lid into which the drop 11 of liquid is deposited.

In operation, the user engages the capillary tube and presses the lid retractor against the lower lid to form the cul-de-sac 24 whereafter the drop 11 of liquid is dispensed from the dispenser and deposited into the cul-de-sac. An interior surface 25 of a channel 26 of the capillary tube is smooth and calibrated so that the liquid can easily travel thereon. A coating can be applied to the inner surface to ensure the smooth travel of the liquid, as for example, by applying a Teflon™ coating on the interior surface 25. A one-way valve 27 is provided in the capillary channel in proximity to the juncture of the capillary channel with the receiver 13 in order to prevent reflux of the liquid back into the receiver. The one-way valve 27 can be constructed as a simple flap valve which will allow passage of the liquid into the capillary channel but prevent reverse flow out of the channel.

Preferably, the instrument 10 is made of an inexpensive plastic material so that it can be disposed of even after a single use.

The construction of the described instrument 10, enables the user to deposit the drop of liquid into his or her eye without any need for the user to occupy any special position in order to dispense the liquid drop. In this respect, it is unnecessary for the user to tilt his or her head back or to lie in a supine position in order to administer the drop. The instrument is simple to use such that the liquid drop will be precisely deposited into the user's eye using one hand of the user. In this respect, when the lid retractor is pressed in proximity to the lower lid of the eye, the discharge outlet 17 of the capillary tube will automatically be positioned to deposit the drop of liquid into the cul-de-sac of the eye. It is contemplated that a drop of liquid will be dispensed from the dispenser 14 into the receiver 13 and the user will have adequate time to retract the lower lid and to dispense the liquid drop into the cul-de-sac of the eye. Alternatively, the liquid from the dispenser can be deposited into the receiver after the lid retractor has been pressed against the lower lid.

Because of the low cost of the instrument, a number of instruments can be included in a package and removed one by one for individual use.

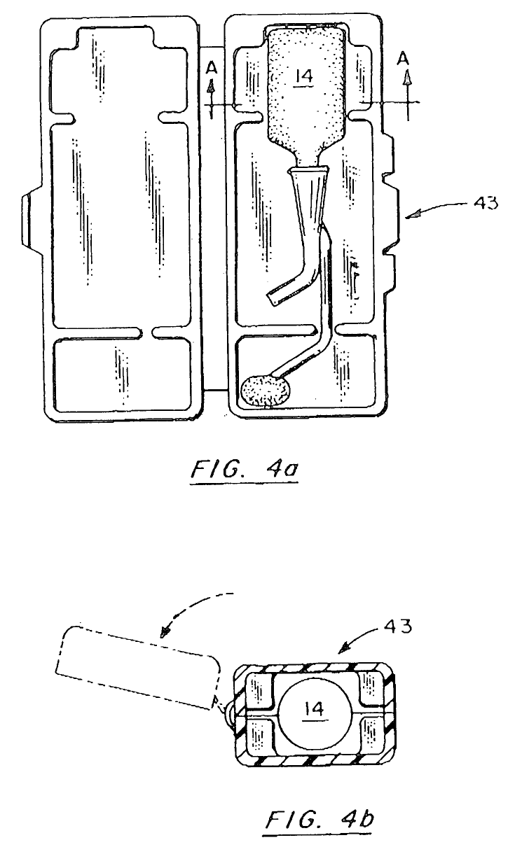

The instrument of FIGS. 1-3 is adapted to be used for dispensing a multitude of drops until the contents of the dispenser 14 are exhausted. Therefore, a cover may be in the form of a case 43 utilized to preserve sanitary conditions between the dispensing of drops. Referring to FIGS. 4a-4b, the case is internally configured to receive both the instrument 10 and the dispenser 14 while they are maintained in a coupled pre-dispensing relationship. The case 43 is of a size to enclose the entire instrument therewithin as shown in FIGS. 4a-4b. When the instrument is to be used to dispense a drop of liquid into the user's eye, the cover 43 is opened, the instrument 10 and dispenser 14 are removed as a unit, the lid retractor is pressed against the lower lid, and the dispenser 14 is squeezed to discharge a drop of liquid into the instrument for discharge at outlet 17. The instrument and dispenser are then replaced in the case which is pivotally closed in the FIG. 4b arrow direction to maintain sanitary conditions between individual dispensing of drops. The case 43 can also be color coded or otherwise identified to indicate the contents of the dispenser. The cover or case can also assume a number of other forms.

The configuration of the discharge outlet is intended to ensure formation of the drop of liquid and its discharge into the eye of the user. Other modified forms of the discharge outlet are also within the contemplation of the invention provided they form and discharge the drop of liquid into the eye of the user.

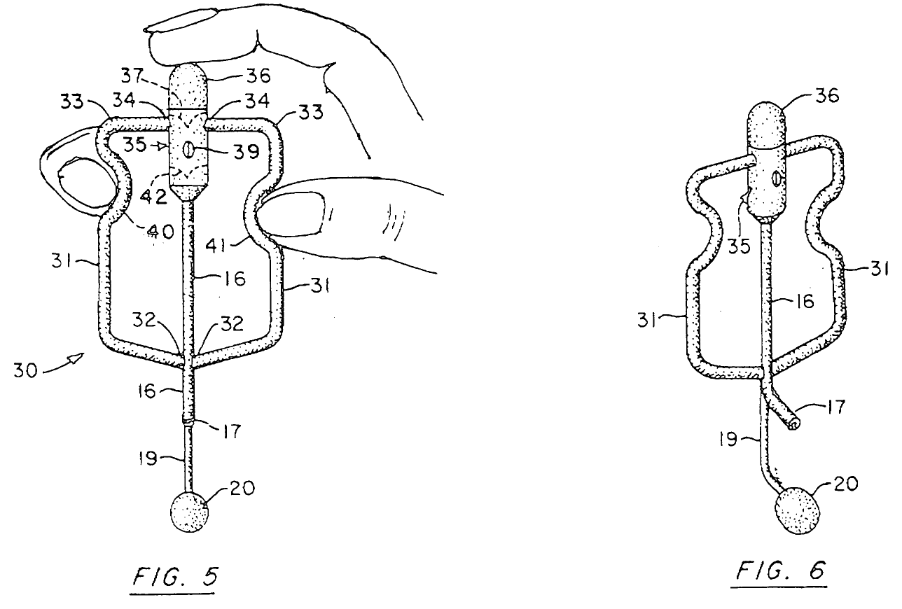

FIGS. 5-6 show another embodiment, embodiment two, of the invention in which the same or similar elements in FIGS. 1-3 have the same reference numerals.

The embodiment two of FIGS. 5-6 employs a holder 30 which enables the user to retract the lower lid and dispense a drop of liquid with a one-hand operation. The holder 30 comprises two rod members 31 whose lower ends 32 are fixed to the capillary tube 16. At their upper ends the rods are bent to form portions 33 which are attached to the loading chamber at site 34. At the upper end of the capillary tube 16, there is secured a loading chamber 35 which replaces the receiver 13 in the embodiment one of FIGS. 1-3. The loading chamber 35 is connected to a deformable air-filled bulb 36 which projects beyond the bent portions 33 of the holder so that a portion of the bulb is compressed by the fingers to dispense the liquid, as will be explained subsequently. The loading chamber 35 incorporates flap valve 37 which defines the upper end of the loading chamber 35 and another flap valve 42 defines the lower end of the loading chamber. In addition, there is a cover valve 39 into which a nozzle of a dispenser (not shown) can be introduced in order to fill the loading chamber 35 with liquid.

The rods 31 are provided with internally dimpled depressions 40 and 41 which form finger-engaging recesses. A second one-way valve 42 is provided at the bottom of the loading chamber 35 at the juncture thereof with the capillary tube 16 to prevent reflux of liquid from the capillary tube.

In use, the user engages the finger-engageable portion 41 with his or her thumb and the finger-engaging portion 40 with his or her middle finger. The soft material of the pressing member 20 is then pressed against the lower lid of the eye and the flexible bulb 36 is pressed with the forefinger of the user to cause a drop of the liquid to pass from the loading chamber 35 into the capillary tube 16 to the outlet thereof to be dispensed into the cul-de-sac 24 of the lower lid.

As shown in FIG. 5, the rods 31 are rectilinear and lie in a common plane to form a flat profile for the holder 30 which is coplanar with the capillary tube.

In the embodiment two of FIG. 6, rod 19 of the lid retractor is directly connected to the bottom of the bent portion of the capillary tube.

As seen in the above, the invention provides a hand-engageable instrument for administering eye drops to the eye of the user, in which the transfer section forms a liquid flow means for dispensing the liquid one drop at a time to the discharge outlet thereof in combination with the lid retractor which exposes the cul-de-sac at the lower lid into which the liquid drop is deposited. The liquid flow portion and the lid retractor portion of the device are integrated and arranged so that the discharge outlet of the flow system is positioned to deposit the drop of liquid directly and accurately into the cul-de-sac when the lid retractor is pressed against the lower lid. The lid retractor and the liquid flow portion are so integrated and arranged that the instrument can be manually held in one hand for dispensing the liquid drop into the eye.

Although the invention is disclosed with reference to particular embodiments thereof, it will become apparent to those skilled in the art that numerous modifications and variations can be made which will fall within the scope and spirit of the invention as defined by the attached claims. For example, the invention may also be an inseparable part of the dispensing bottle itself so that the dispenser and bottle are a single piece. This would still be within the scope and spirit of the invention as defined by the attached claims.

1. A system for delivering a single drop of liquid to an eye of a subject, said system comprising:
a receiver adapted to receive a liquid to be delivered to an eye of a subject.
a transfer portion connected to said receiver to receive liquid therefrom, said transfer portion including a capillary tube to advance a drop of said liquid by gravity and capillary action to a lower discharge outlet of the capillary tube whereat the drop of liquid breaks away and drops from the capillary tube, and
a lid retractor supported adjacent to said discharge outlet of the capillary tube, said lid retractor being adapted for being pressed against the lower lid of the subject to form a cul-de-sac between the lower lid and the eye,
said lower discharge outlet of said capillary tube being positioned adjacent to and above said lid retractor in a position to deposit the drop of liquid into the cul-de-sac.

2. The system of claim 1, wherein said receiver has a funnel shape for attaching to and receiving the liquid from a container containing the liquid.

3. The system of claim 1, wherein said capillary tube has a smooth inner surface along which the drop of liquid travels.

4. The system of claim 1, further comprising a one way valve between said receiver and said capillary tube which permits passage into said capillary tube of said drop of liquid and prevents reflux of the liquid from the capillary tube back into the receiver.

5. The system of claim 1, wherein said capillary tube has a lower end portion which is bent at an angle and is above said lid retractor.

6. The system of claim 1, wherein said lid retractor has a soft substance for contacting skin of the subject.

7. The system of claim 1, wherein said receiver, said transfer portion and said lid retractor are made of a low cost material.

8. The system of claim 7, wherein said low cost material is a plastic.

9. The system of claim 1, wherein said receiver has an upper end portion formed as a flexible bulb connected to a loading chamber.

10. The system of claim 9, further comprising finger engageable portions secured to said transfer portion to enable the subject to engage the finger engageable portions with a thumb and middle finger of one hand and press the bulb with a forefinger of the same hand to expel liquid from the device.

11. The system of claim 10, wherein said loading chamber has a valve opening for depositing liquid from a container into the loading chamber.

12. The system of claim 10, wherein said finger engageable portions include two rod members on opposite sides of said capillary tube, each rod member having a recess for engagement by a finger of the subject.

13. The system of claim 12, wherein said rod members are secured to said capillary tube.

14. The system of claim 12, wherein said rod members have lower ends fixed to said capillary tube and bent upper ends facing one another which is connected to the loading chamber.

15. The system of claim 1, wherein said capillary tube has an end portion provided with said lower discharge outlet, said end portion of said capillary tube being of conical shape.

16. The system of claim 1, comprising an extension on said capillary tube at said discharge outlet to guide flow of said drop of liquid to the eye of the subject.

17. The system of claim 1, wherein said receiver has a flexible upper end shaped and dimensioned to grip a tip of a container of said liquid inserted therein.

18. The system of claim 17, comprising a sterile case sized to enclose said receiver, said transfer portion and said lid retractor.

19. A hand engageable device for administering eye drops to an eye of a subject, said device comprising liquid flow means for transporting liquid, one drop at a time, to a discharge outlet of the liquid flow means and lid retractor means adapted for being pressed against a lower lid of an eye of the subject to produce a cul-de-sac of the lower lid, said liquid flow means and said lid retractor means being integrated and arranged so that said discharge outlet is positioned to deposit a drop of liquid into the cul-de-sac when said lid retractor means is pressed against the lower lid.

20. The device of claim 19, wherein the liquid flow means comprises a capillary tube.

21. The device of claim 20, further comprising a receiver for receiving the liquid to be deposited as drops into the eye of the subject, said receiver being connected to said capillary tube to deliver said drop of liquid thereto.

22. The device of claim 20, wherein said liquid flow means comprises a one-way valve for passing said drop of liquid to the capillary tube.

23. Apparatus for depositing a drop of liquid into an eye of a subject, said apparatus comprising a manually held instrument including a lid retractor and a liquid conveyor integrated and arranged so that liquid introduced into the liquid conveyor is advanced as a drop and deposited into an eye of a subject whose lower lid is retracted by the lid retractor.

24. The apparatus of claim 23, wherein said liquid conveyor comprises a capillary tube.

25. The apparatus of claim 24, wherein the liquid conveyor comprises a receiver to receive a drop of liquid, said receiver being connected to said capillary tube to deliver said drop of liquid thereto.

26. The apparatus of claim 24, wherein said liquid conveyor comprises a one-way valve for passing said drop of liquid to the capillary tube.

27. A method of depositing a drop of liquid into an eye of a subject comprising:
providing an instrument adapted for being held in one hand of the subject,
pressing, with said instrument, a lower lid of the subject to form a cul-de-sac of the lower lid, and
advancing a drop of liquid in the instrument to a discharge end thereof wherefrom the drop of liquid falls into the cul-de-sac.

28. The method of claim 27, wherein first the instrument is pressed against the lower lid of the subject to expose the cul-de-sac whereafter the liquid is introduced into the instrument and the drop of liquid is guided and advanced to the discharge end where it drops into the cul-de-sac.
